(19) United States
(12) Patent Application Publication
(10) Pub. No.: US 2025/0295888 A1
(43) Pub. Date: Sep. 25, 2025

(54) MULTI-DIRECTIONAL FLOW CATHETER

(71) Applicant: Aegis Medical Technologies, LLC., Philadelphia, PA (US)

(72) Inventor: Timothy William Ingraham Clark, Philadelphia, PA (US)

(21) Appl. No.: 18/905,495

(22) Filed: Oct. 3, 2024

Related U.S. Application Data
(60) Provisional application No. 63/569,425, filed on Mar. 25, 2024.

(51) Int. Cl. A61M 25/00

(52) U.S. Cl. CPC A61M 25/003; A61M 25/001; A61M 25/007; A61M 25/0071; A61M 2025/0031; A61M 2025/0037; A61M 2025/0073; A61M 2202/0021; A61M 2202/0413; A61M 2206/16

(57) ABSTRACT

Disclosed are catheters useful in dialysis treatment of a patient. In an example, a catheter includes a tip that has an arterial lumen that directs blood flow in the direction of a dialysis machine and a venous lumen that directs flow in a direction away from the dialysis machine. One or both of the arterial and venous lumens includes within it a spiral flow inducing structure that cause blood flow to rotate as it travels through the lumen. In particular applications the one or more spiral flow inducing structure are spiral laminar flow inducing structures.

### CROSS-REFERENCE TO RELATED APPLICATIONS

This application relates to and claims the benefit and priority to Provisional Patent Application. No. 63/569,425, filed Mar. 25, 2024.

### FIELD

The present invention relates to the field of flow delivery catheters, and in particular to multi-directional flow catheters that are configured to induce spiral fluid flows in at least two flow directions of a fluid.

### BACKGROUND

Over 600,000 Americans have end-stage kidney disease and require renal replacement therapy to sustain life. For most of these patients, this therapy is hemodialysis which involves removal of blood during a hemodialysis session and returning the blood after balancing solutes and electrolytes across an exchange membrane within the blood flow circuit. In the United States, dialysis catheters are used for initial vascular access for over 80% of patients initiating dialysis each year and serve as a vital bridge to a surgically or endovascularly created arteriovenous fistula or arteriovenous graft. Dialysis catheters are also used as a vascular access lifeline when patients have depleted prior fistulas or grafts due to thrombosis, infection, or other vascular complications.

Current dialysis catheters are capable of high rates of blood flow (300-400 mL/min) required for efficient hemodialysis. Each session of hemodialysis is 3-4 hours in length for intermittent hemodialysis, 8 hours in length for nocturnal hemodialysis, and nearly continuous for certain critically ill patients. The high rates of blood flow during hemodialysis create elevated shear forces on blood cells entering and exiting the lumens of the hemodialysis catheter, which has the potential to produce hemolysis of red blood cells and platelet activation. Dialysis catheters are distinguished from each other by the catheter tip(s), where all fluid intake and outflow occurs and the region prone to the greatest shear stress and nonlaminar flow patterns.

In the discussion that follows, fluid flow in a direction towards the inlet of the dialysis machine is referred to as “arterial fluid flow”, and fluid flow in a direction away from the outlet of the dialysis machine is referred to as “venous fluid flow”.

Spiral laminar flow is a natural phenomenon within the human vascular system which occurs at areas of blood vessel branching and high rates of blood flow. Spiral laminar flow creates helical vortices of blood which are less likely to lead to platelet adherence and aggregation, red blood cell hemolysis, or leukocyte adhesion. These phenomena contribute to thrombus formation within the catheter lumens, impeding flow and preventing effective dialysis. Most dialysis catheters have tip designs which create little to no spiral laminar flow, and simply rely on high rates of blood flow achieved with larger lumen sizes. A dialysis catheter design which creates spiral laminar flow within the ‘arterial’ (toward the dialysis machine) and ‘venous’ (returning from the dialysis machine) lumens would be less likely to experience catheter failure from thrombus formation.

Another important parameter of dialysis catheter performance for optimal solute clearance is recirculation of dialyzed blood through the dialysis circuit through the admixture of ‘venous’ and ‘arterial’ blood. Higher rates of recirculation require longer durations of hemodialysis for target solute removal or blood pressure control. Strategies to reduce recirculation in dialysis catheter design have predominately utilized spatial separation of intake and output ports. However, as the number of patients on hemodialysis continues to grow with a greater spectrum of vascular anatomy of the superior vena cava and right atrium, spatial separation of catheter intake and output ports may be inadequate if the surrounding blood vessel is narrowed due to disease and/or scar tissue. Spiral laminar flow of fluid entering and exiting the dialysis catheter has the potential to minimize recirculation by aligning the vortices in opposing directions.

Prior solutions to creating spiral directions of flow within fluid entering (arterial flow) and exiting (venous flow) the lumens of a multi-lumen catheter have included curved walls defining the terminal/distal ends of apertures through which fluid enters or exits the catheter (U.S. Pat. No. 7,569,029). A limitation of these solutions is that venous fluid flow does not assume a spiral direction of flow until it encounters a curved wall at the distal end of the venous flow output port of the catheter. Another limitation of these solutions is that arterial fluid flow entering the inlet port of the arterial lumen of the catheter must change flow direction rapidly as it flows across the curved distal aperture of the catheter, so that platelet cell activation may occur with subsequent adherence and aggregation to the catheter surface. Another solution describes a spiral twist of the central septum separating the first and second lumens of the catheter (U.S. Pat. No. 10,220,184).

Spiral laminar flow interfaces have also been integrated into the design of a vascular bypass conduit in the form of helical ridges or grooves within the tubing wall of the graft (U.S. Pat. No. 9,737,421). A limitation of this solution is it cannot be applied to the non-circular shape of a dialysis catheter lumen.

### SUMMARY

Disclosed are multi-directional catheters that may be used in a variety of applications. One particular use is in the dialysis treatment of patients. Disclosed herein are dialysis catheters that have a proximal end portion that is configured to be coupled to the dialysis machine to deliver to and retrieve blood from the dialysis machine. The dialysis catheters also include a distal end section/tip through which blood enters and exits the catheter when the distal end section resides inside a patient during a dialysis procedure.

The proximal end or end portion of the dialysis catheters disclosed herein are configured to reside outside the body of the patient. According to some implementations the proximal end or proximal end portion of the dialysis catheter includes a first connector connectable to a fluid inlet of the dialysis machine and a second connector connectable to the fluid outlet of the dialysis machine.

According to some implementations the distal end section/tip of the catheter includes an arterial lumen configured to carry blood in a proximal direction towards the dialysis machine. The arterial lumen includes an inlet port located at a distal end or distal end portion thereof and an outlet port located at a proximal end or a proximal end portion thereof. Located inside the arterial lumen at a first longitudinal distance away from the inlet port is a first spiral flow inducing structure that is located entirely inside and surrounded by the wall or walls that form the arterial lumen. According to some implementations the first spiral flow is a spiral laminar flow.

The distal end section of the catheter also includes a venous lumen configured to carry blood in a distal direction away from the dialysis machine. In the tip of the catheter the arterial and venous lumens are respectively formed at least in part by first and second sides of a first septum. According to some implementations, the venous lumen of the catheter tip includes an inlet port located at a proximal end of the tip, and first and second outlet ports that are each located distal to the inlet port. The first and second outlet ports of the venous lumen are circumferentially spaced-apart from one another with each extending through a wall of the tip at a longitudinal location proximal to the distal end of the catheter. Located in the venous lumen is a second spiral flow inducing structure that is configured to direct a first portion of the blood towards the first outlet port and a second portion of the blood to the second outlet port. According to some implementations, the second spiral flow inducing structure is a monolithic structure.

According to some implementations, spiral laminar flow is imparted to a fluid (e.g. blood) flowing through the venous lumen towards the first and second outlet ports. This occurs at a distance proximal to the distal end of the catheter. Advantageously, fluids such as blood are less likely to form obstructions within the venous lumen through the activation of platelet cells with subsequent adherence and aggregation to the venous lumen wall. It is additionally advantageous to impart spiral laminar flow to fluid flowing through the arterial lumen with the spiral laminar flow being induced at a location inside the arterial lumen proximal to the inlet port of the arterial lumen, so that fluids such as blood are less likely to form obstructions within the arterial lumen.

According to other implementations the catheter tip includes a venous lumen configured to carry blood in a distal direction away from the dialysis machine. The venous lumen includes an inlet port, a first outlet port and a second outlet port, the first and second outlet ports being located distal to the inlet port and being circumferentially spaced-apart from one another with each extending through a wall of the catheter. Located in the venous lumen is a first spiral flow inducing structure that includes first and second surfaces that are respectively configured to direct first and second portions of blood flow towards the first and second outlet ports. According to some implementations the first spiral flow inducing structure causes each of the first and second portions of blood to flow in spiral laminar fashion. The catheter also includes an arterial lumen configured to carry the blood in a proximal direction towards the dialysis machine. At least proximal end portions of the venous and arterial lumens located in the catheter tip are respectively formed at least in part by first and second sides of a first septum. According to some implementations, the arterial lumen includes an inlet port located at or near a distal end of the catheter and an outlet port located proximal to the distal end of the catheter. Located inside the arterial lumen at a first longitudinal distance away from the inlet port is a second spiral flow inducing structure that is located entirely inside and surrounded by the wall or walls that form the arterial lumen. According to some implementations, the spiral flow inducing structure induces a spiral laminar flow of the blood.

According to other implementations, the arterial lumen that carries blood towards the dialysis machine comprises side-by-side first and second arterial sub-lumens that are separated by a second septum, the first and second arterial sub-lumens respectively include first and second inlet ports located at or near the distal end of the catheter. The arterial lumen further includes at least one outlet port located proximal to the first and second sub-lumens. Located inside the first arterial sub-lumen at a first longitudinal distance away from the first inlet port is a second spiral flow inducing structure that is located entirely inside and surrounded by the wall or walls that form the first arterial sub-lumen. Located inside the second arterial sub-lumen at or near the first longitudinal distance away from the first inlet port is a third spiral flow inducing structure that is located entirely inside and surrounded by the wall or walls that form the second arterial sub-lumen. According to some implementations, each of the second and third spiral flow inducing structures induces a spiral laminar flow of the blood.

As explained above, blood entering the arterial lumen of a catheter under high flow conditions such as hemodialysis or plasmapheresis is subjected to rapid transitions in the direction of flow. These rapid transitions in flow direction produce turbulence and mechanical shear forces on the cellular elements of blood, particularly the cell membranes of platelets, which in turn become activated and release granules. This repetitive platelet trauma leads to aggregation and adhesion of platelets to the surface of the catheter, which then generates thrombin, leading to deposition of fibrin and other proteins to create obstructive thrombus. This obstruction results in mechanical failure of the catheter and creates a biomatrix for the aggregation of circulating microorganisms to enable infection of the catheter.

When the entry aperture of the arterial lumen is angled, curved or otherwise modified to impart a transition in the direction of flow as blood enters the catheter as disclose in U.S. Pat. No. 7,569,929, this transition occurs over a short distance corresponding to the thickness of the wall of the aperture, e.g. 30/1000ths of an inch or 0.76 mm. Therefore, there remains an opportunity to improve the function of dialysis catheters by utilizing favorable flow deflecting interfaces incorporated into the lumens of a catheter instead of limited to the terminal apertures of the catheter.

According to some implementations an arterial lumen of the present invention has flow deflecting interfaces within the lumen, as opposed to being located at the terminal apertures/inlet ports. These interfaces preferably, but not necessarily, impart spiral laminar flow patterns to blood entering the catheter over a substantially longer distance of the catheter length (approximately 5 to 15 millimeters, compared to less than one millimeter discussed above), thereby further reducing mechanical shear stress on cellular elements of blood, such as platelets. When measured using a validated computational fluid dynamics model described by Nobili et al. (Nobili M, Sheriff J E, Morbiducci U, Redaelli A, Jesty J, Bluestein D. Platelet activation due to hemodynamic shear stresses: damage accumulation model and comparison to in vitro measurements. ASAIO J 2008; 54:64-72.), according to some implementations average platelet activation states of 1.4×10⁻⁶ are achieved. This catheter thrombosis risk is substantially lower than currently available dialysis catheters.

The venous lumen of the present invention has flow deflecting interfaces within the lumen as opposed to the prior art where they are located only at the terminal apertures. In addition to producing less shear stress on blood cells exiting the catheter, the flow deflecting interfaces within the lumen also exert directionality to blood so that blood flows away from the catheter (the blood flow comprising a radial component) to minimize the possibility of recirculation of dialyzed blood into the arterial inlets of the catheter at the tip of the catheter. Using a validated computational fluid dynamics model, according to some implementations a calculated recirculation percentage of 0.002% is achieved, meaning that 99.998% of dialyzed blood leaving the catheter does not reenter the arterial lumens. In contrast, many current dialysis catheters exhibit recirculation percentages of 10-30%. These high levels of recirculation reduce the adequacy of dialysis and results in less efficient clearance of toxins from the bloodstream. Inadequate dialysis is a contributing factor to increased hospitalizations, hospital days and inpatient expenditures among dialysis patients. Therefore, it is clinically advantageous to have a catheter with minimal or absent recirculation.

These and other advantages and features of will become apparent in view of the figures and detailed description.

### BRIEF DESCRIPTION OF THE DRAWINGS

FIG. 1A shows a dialysis catheter according to one implementation.

FIG. 1B shows a cross-section view of the catheter of FIG. 1 along line A-A.

FIG. 2 illustrates a tip of a dialysis catheter according to one implementation with blood flow flowing into an arterial lumen of the catheter and blood flow flowing out of a venous lumen of the catheter through two exit ports.

FIG. 3 shows another view of the tip of the dialysis catheter of FIG. 2.

FIG. 4 shows another view of the tip of the dialysis catheter of FIG. 2.

FIG. 5 shows another view of the tip of the dialysis catheter of FIG. 2.

FIG. 6 illustrates a tip of a dialysis catheter according to another implementation with blood flow flowing into an arterial lumen of the catheter and blood flow flowing out of a venous lumen of the catheter through three exit ports.

FIG. 7 shows another view of the tip of the dialysis catheter of FIG. 6.

FIG. 8. Illustrates blood flow into, through and out of an arterial lumen of a dialysis catheter tip according to one implementation.

FIG. 9 is a longitudinal cut-away view of a dialysis catheter tip according to one implementation, wherein the arterial lumen is shown.

FIG. 10 is a longitudinal cut-away view of a dialysis catheter tip according to another implementation, wherein the arterial lumen is shown.

FIG. 11 is a proximal facing view of the dialysis catheter tip of FIG. 9.

FIG. 12 is an isometric view of a curved ramp section in arterial sub-lumen of a dialysis catheter tip according to one implementation.

FIG. 13 is a longitudinal cross-section view of a dialysis catheter tip according to one implementation.

FIG. 14 is a distal facing view of a dialysis catheter tip according to one implementation.

FIG. 15 is a partial cutaway view of the dialysis catheter tip of FIG. 14

### DETAILED DESCRIPTION

The present invention is directed to multi-directional catheters that may be used in a variety of applications. In the examples that follow, multi-directional catheters in the form of dialysis catheters are disclosed. It is appreciated, however, that the invention can be implemented in catheters other than dialysis catheters.

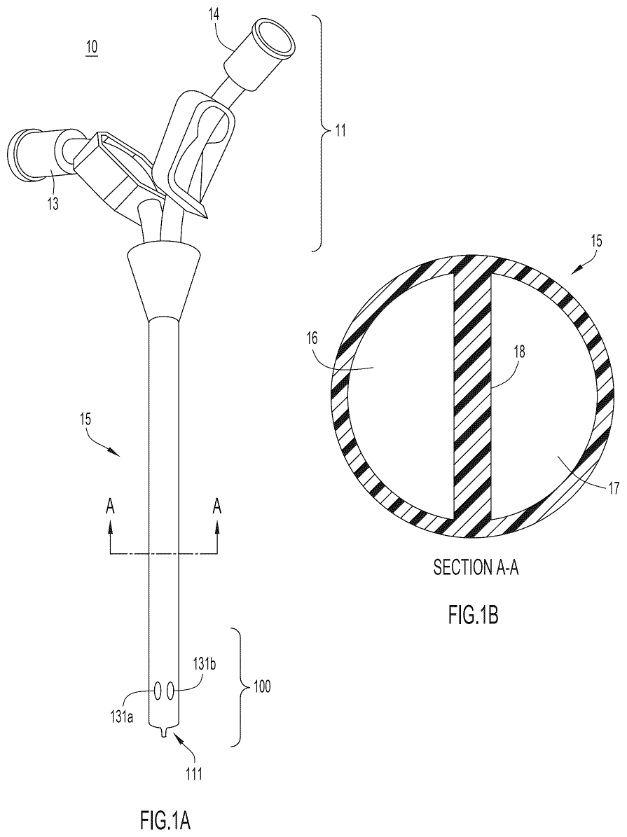

FIGS. 1A and 1B illustrate a dialysis catheter 10 that has a proximal end portion 11 that is configured to be coupled to a dialysis machine (not shown) to deliver to and retrieve blood from the dialysis machine. The dialysis catheter 10 also include a distal end section 100 (also referred to herein as a “tip”) through which blood enters and exits the catheter when the distal end section resides inside a patient during a dialysis procedure.

The proximal end portion 11 of the dialysis catheter is configured to reside outside the body of the patient at all times. The proximal end portion 11 of the dialysis catheter 10 includes a first connector 13 connectable to a fluid inlet of the dialysis machine (not shown) and a second connector 14 connectable to the fluid outlet of the dialysis machine. The catheter body 15 includes side-by-side arterial and venous lumens 16 and 17 that are separated by a septum 18 extending longitudinally along an internal length of the catheter body 15. The arterial lumen 16 is in fluid communication with the first connector 13 and is configured to direct blood from inside the patient towards the dialysis machine. The venous lumen 17 is in fluid communication with the second connector 14 and is configured to direct blood away from dialysis machine and back into the patient.

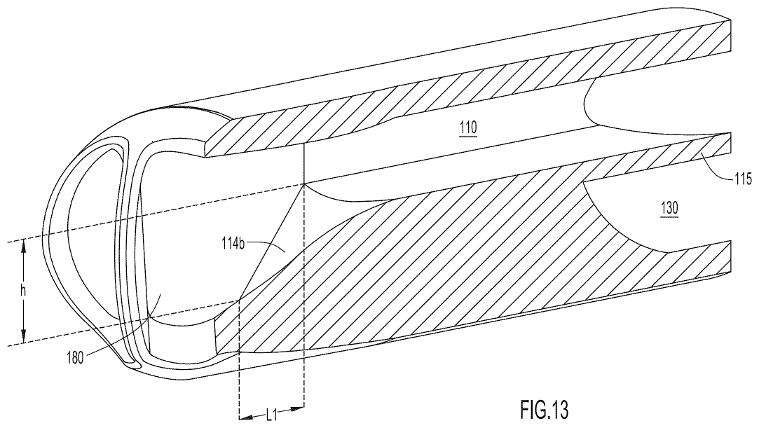

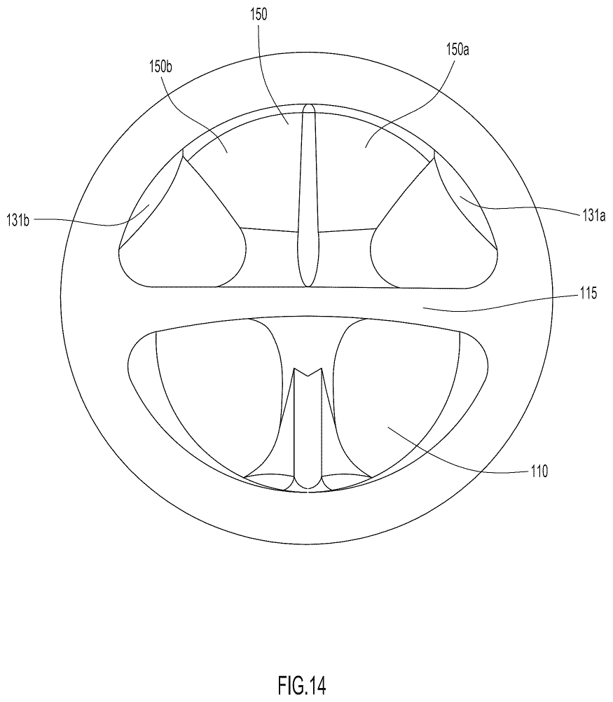

The arterial and venous lumens 16 and 17 are respectively in fluid communication with arterial and venous lumens 110 and 130 of the catheter tip 100. Blood flows in a distal direction “x” through the venous lumen 130 and flows in a proximal direction “y” through the arterial lumen 110. As will be discussed in more detail below, according to some implementations the arterial lumen 110 and venous lumen 130 of the tip 100 are not arranged side-by-side along the entire length of the tip for reason that the venous lumen 130 distally terminates in the tip proximal to the inlet port 111 of the arterial lumen 110 as best shown in FIGS. 13 and 14. Inside the catheter tip 100 where the arterial lumen 110 and venous lumen are arranged side-by-side, they are separated by a first septum 115. Further, according to some implementation the tip 100 is a monolithic structure being made of a single piece of material. For example, tip 100 may be made of a polymeric material through the use of an extrusion process, a 3-D printing process and/or a molding process.

In the disclosure that follows, focus is directed to the tip 100 of the catheter 10 wherein, according to some implementations, spiral flow (and according to some implementations, spiral laminar flow) is imparted to the blood flowing through the venous lumen 130 towards outlet ports 131a and 131b located proximal to the distal end 101 of the tip. This is done so that the blood is less likely to form obstructions within the venous lumen through the activation of platelet cells with subsequent adherence and aggregation to the lumen wall. Further, according to some implementations, spiral flow (preferably spiral laminar flow) is imparted to blood flowing inside the arterial lumen 110 at a location proximal to its inlet port 111. Like with the venous lumen 130 discussed above, this is done so that the blood is less likely to form obstructions within the arterial lumen. Further, the inducement of spiral laminar flow at a location inside the arterial lumen proximal to its inlet port 111 beneficially reduces sharp bending of the blood flow at the inlet port. As explained above, this beneficially reduces platelet cell activation when blood flows into the arterial lumen 110.

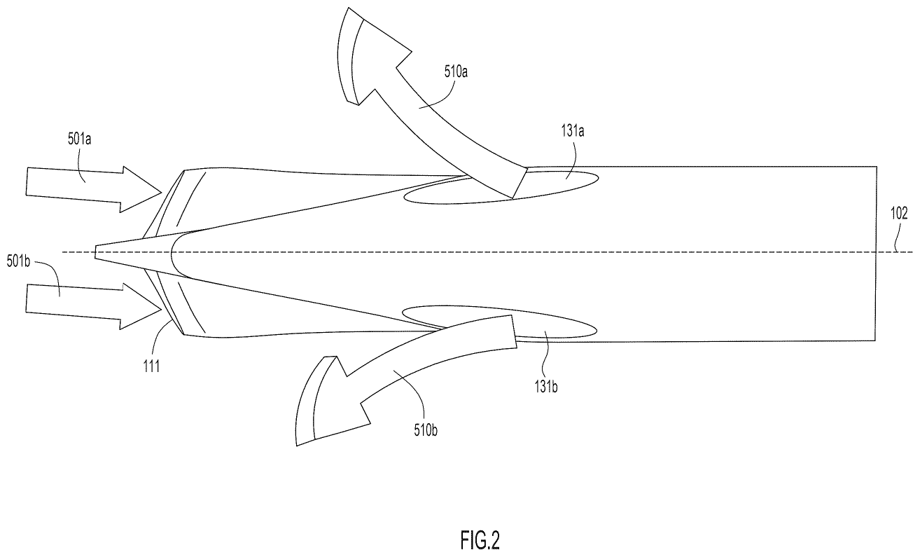

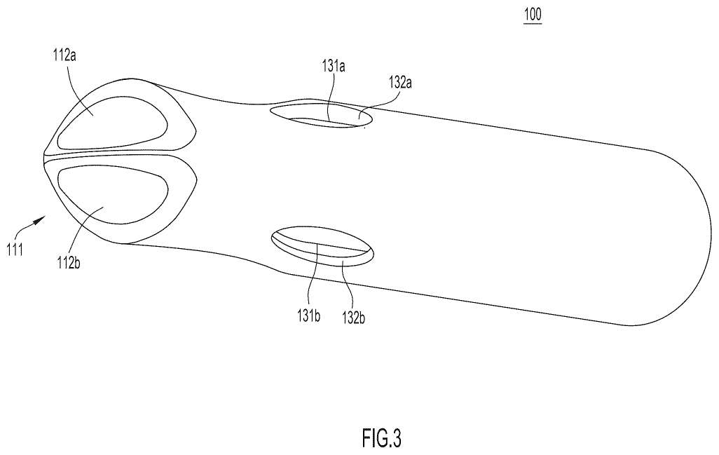

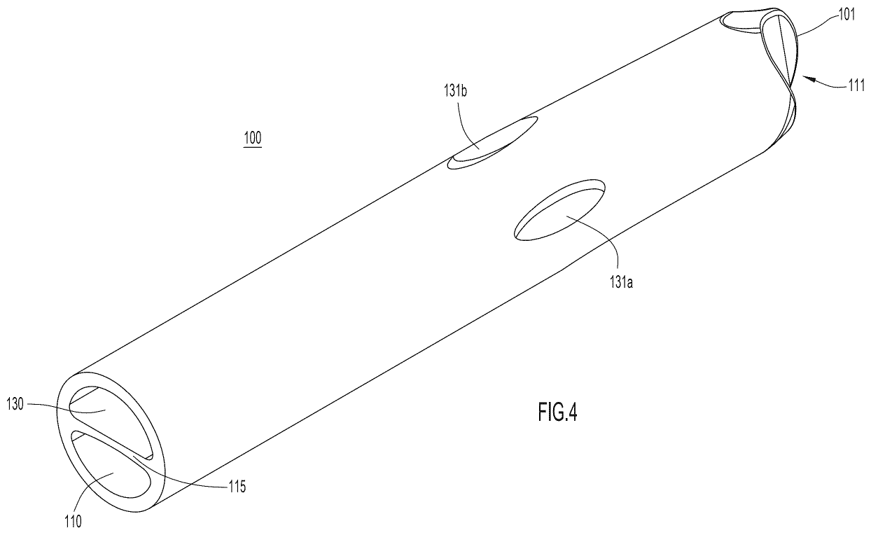

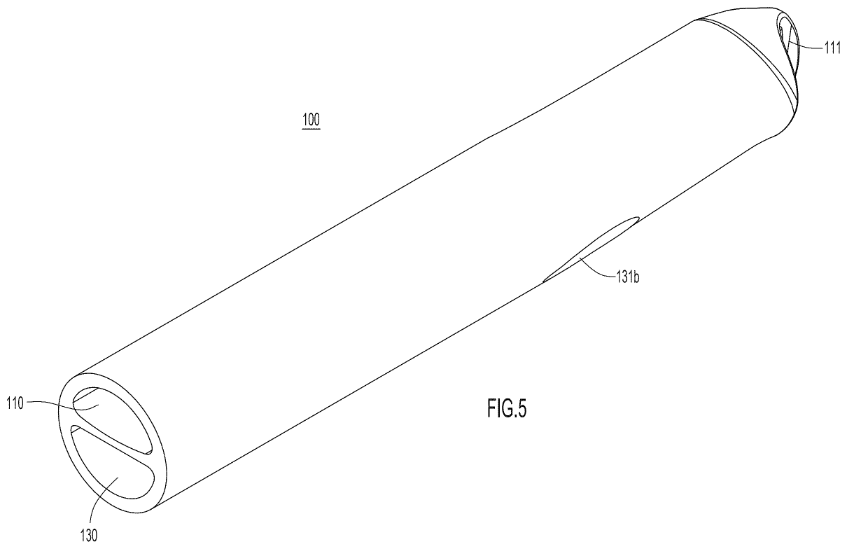

FIG. 3 illustrates a dialysis catheter tip 100 according to one implementation wherein the venous lumen 130 includes two outlet ports 131a and 131b through which spiral flows of blood 510a and 510b exit the tip. According to some implementations these spiral flows of blood are spiral laminar flows of blood. As will be discussed in more detail below, the tip 100 includes features inside the venous lumen 130 that function to create the two spiral flows or spiral laminar flows. The walls 132a and 132b of the outlet ports 131a and 131b may also be configured to promote the formation of spiral flow or spiral laminar flow. In the implementation of FIG. 2, the tip 100 is configured to produce flows 510a and 510b with vortices in opposing directions (and non-parallel to a longitudinal axis 102 of the tip 100 for the purpose of minimizing recirculation into the inlet port 111 of the arterial lumen 110.

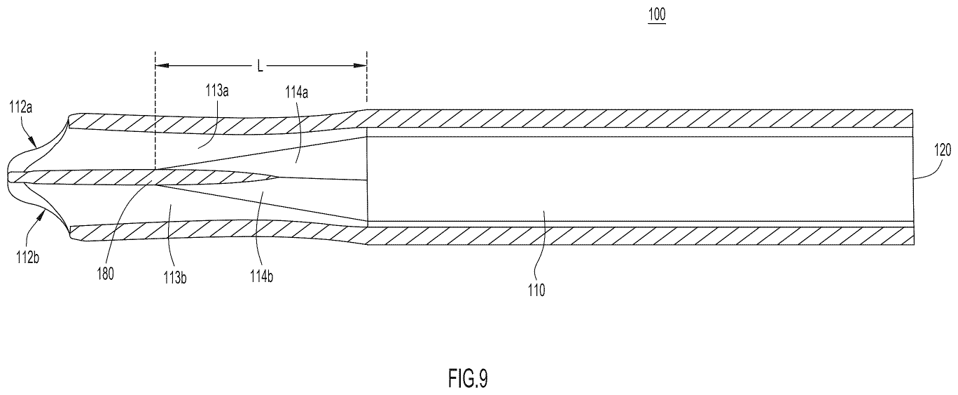

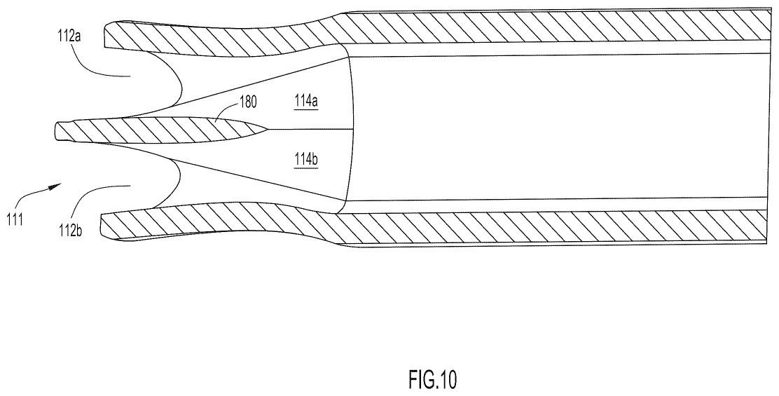

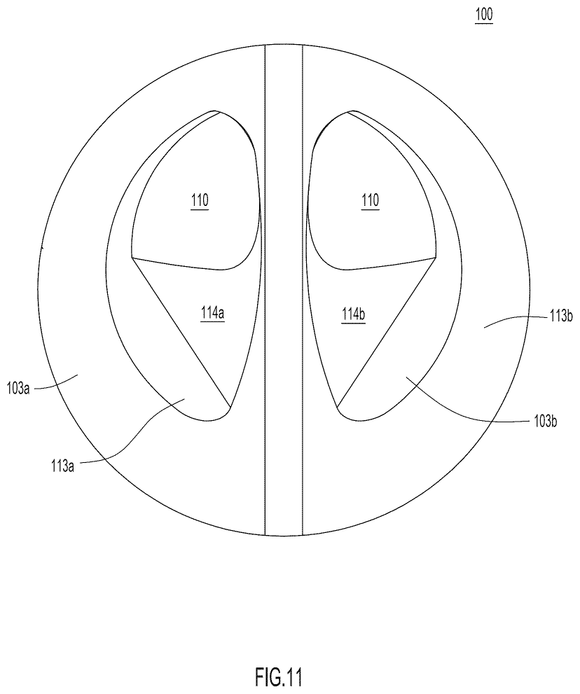

According to some implementations, as shown in FIGS. 9 and 10, the arterial lumen inlet port 111 comprises side-by-side first and second inlet ports 112a and 112b that respectively feed into first and second side-by-side arterial sub-lumens 113a and 113b that are separated at least in part by a second septum 180. According to some implementations the first septum 115 and the second septum 180 are arranged orthogonal with respect to one another. Each of sub-lumens 113a and 113b respectively possesses a first ramped and curved surface 114a and a second ramped and curved surface 114b, each of which is configured to induce spiral flow (and according to some implementations, spiral laminar flow) before the blood flows proximally into the proximal end portion of the arterial lumen 110. The proximal end portion of the arterial lumen being that portion of the arterial lumen 110 that is arranged side-by-side with the venous lumen 130 and is separated therewith by septum 115.

As shown in FIGS. 9 and 10, according to some implementations the surface area of each of the curved ramps 114a and 114b increases as it extends proximally. In other words, the proximal ends of the curved ramps are narrower than their distal ends.

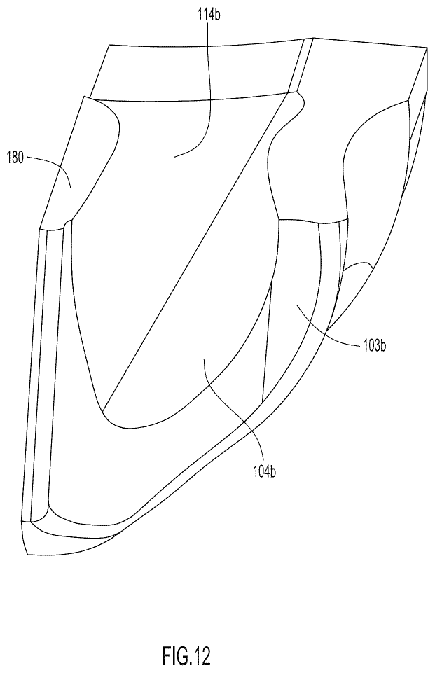

FIG. 12 depicts an example ramped and curved surface 114b that is at least partially formed by the second septum 180 or by a structure that emanates from the second septum. As shown in FIG. 12, according to some implementations surface 114b forms a part of a channel that is also formed in part by a curved inner surface 104b of a wall 103b of the tip 100. With the tip 100 oriented as shown in FIGS. 9 and 13, surface 114b ramps upward by a height “h” of 1.3 to 1.7 millimeters and has a length “L” from 5 to 15 millimeters and preferably between 8 to 12 millimeters. As to the ramped and curved surface 114a located in arterial sub-lumen 113a, according to some implementations it is a mirror image of surface 114b.

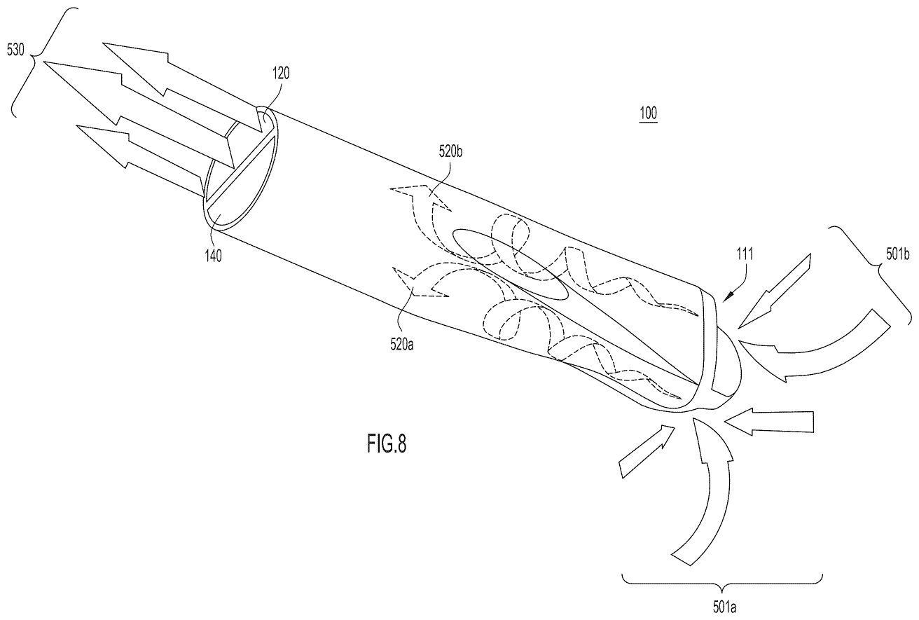

As shown in FIG. 9, according to some implementations blood 501a and 501b from sides of the tip 100 is caused to flow respectively into the inlet ports 112a and 112b of arterial sub-lumens 113a and 113b when, for example, a dialysis treatment is initiated. In implementations wherein surfaces 114a and 114b are mirror images of one another, spiral blood flows 520a and 520b rotate in opposite directions as shown in FIG. 8. As noted above, according to some implementations surfaces 114a and 114b are configured to induce spiral laminar flows inside the respective arterial sub-lumens 113a and 113b. Blood from each of the arterial sub-lumens advances proximally through the tip 100 and collectively exits the arterial lumen 110 outlet port 120 as indicated by arrows 530.

In the implementation of FIG. 9, each of surfaces 114a and 114b is respectively located entirely inside the catheter tip 100 and surrounded by the wall or walls that form the first and second arterial sub-lumens 113a and 113b. However, according to other implementations, as shown in FIG. 10, a distal portion of each of surfaces 114a and 114b may respectively reside outside arterial sub-lumens 113a and 113b.

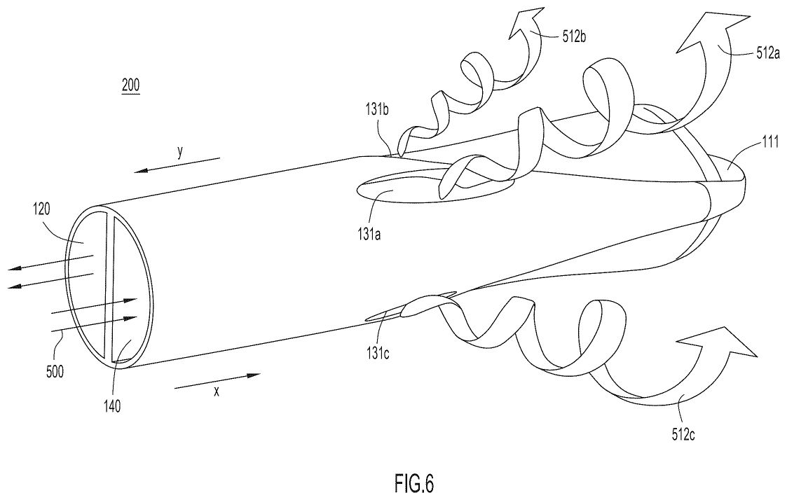

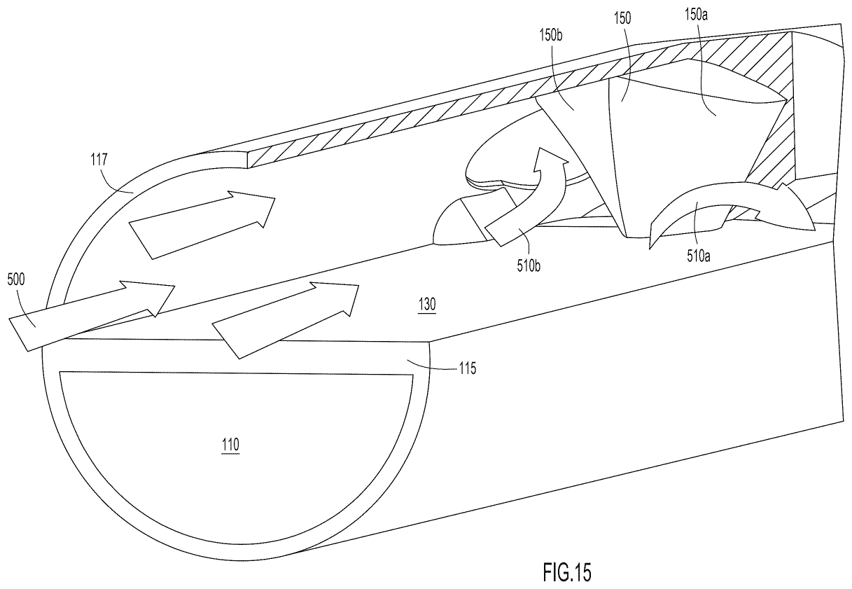

As explained above, the venous lumen 130 is configured to carry blood 500 in a distal direction away from the dialysis machine as illustrated in FIGS. 6 and 15. The venous lumen 130 includes an inlet port 140, a first outlet port 131a and a second outlet port 131b. The first and second outlet ports 131a and 131b are each located distal to the inlet port 140 and are circumferentially spaced-apart from one another with each extending through a wall 117 of the catheter tip 100. According to some implementations the first and second outlet ports 131a and 131b are circumferentially aligned and are angular spaced apart from one another by greater than 90 degrees.

With reference to FIG. 15, located in the venous lumen 130 is a spiral flow inducing structure 150 that includes first and second surfaces 150a and 150b that are respectively configured to direct first and second portions of blood flow 510a and 510b towards the first and second outlet ports 131a and 131b. According to some implementations the spiral flow inducing structure 150 is a monolithic structure made frame a single piece of material. According to some implementations, each of surfaces 150a and 150b is configured to cause spiral blood flows 510a and 510b to be laminar flows. According to some implementations surfaces 150a and 150b are curved to impart a transition in the direction of blood flow. According to some implementations spiral flow inducing structure 150 is shaped like a bow of a ship with each of surfaces 150a and 150b comprising a concave surface. According to some implementations the spiral flow inducing structure 150 is configured to cause the first portion of blood 510a to spiral in a first rotational direction and to cause the second portion of blood 510b to spiral in a second rotational direction opposite the first rotational direction. According to some implementations the spiral flow inducing structure 150 extends between an inner wall of the venous lumen 130 and the septum 115 separates at least a portion of a longitudinal length of the arterial and venous lumens.

As noted above, according to some implementations at least a portion of a first structure 132a that forms the first outlet port 131a is configured to induce spiral flow as the first portion of blood passes through the first outlet port, and at least a portion of a second structure 132b that forms the second outlet port 131b is configured to induce spiral flow as the second portion of the blood passes through the second outlet port.

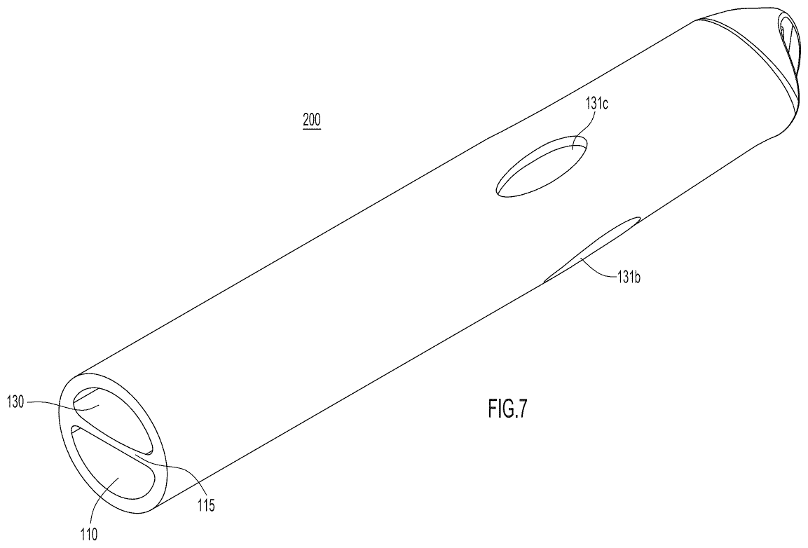

FIGS. 6 and 7 illustrate a dialysis catheter tip 200 according to one implementation wherein the venous lumen 130 includes three outlet ports 131a, 131b and 131c through which spiral flows or spiral laminar flows of blood 512a, 512b and 512c exit the tip with vortices in the same direction. According to other implementations at least two of the three blood flows 512a-c have vortices in opposing directions. The tip 200 includes internal features that function to create the three flows 512a-c. The walls of the outlet ports 131a, 131b and 131c may also be configured to promote the formation of spiral flow or spiral laminar flow. Blood flow is routed to the third outlet port 131c through a conduit (not shown) that extends from the venous lumen 130 and radially across the arterial lumen 110.

According to some implementations spiral flow or spiral laminar flow is achieved as blood inside the human patient flows across the spiral flow inducing or spiral laminar flow inducing structures at a rate of 200-600 mL/min.

Additional implementations are described in the clauses that follow.

### Clause Set A:

Clause 1. A catheter for placement within a vessel or cavity of a body, the catheter comprising:
a tip including:
   a longitudinal axis,
   a first lumen for carrying a fluid in a proximal direction, the first lumen including an inlet port located at a distal end or distal end portion thereof and an outlet port located at a proximal end or a proximal end portion thereof, located inside the first lumen at a first longitudinal distance away from the inlet port is a first spiral flow or spiral laminar flow inducing structure that is located entirely inside and surrounded by the wall or walls that form the first lumen;
   a second lumen for carrying the fluid in a distal direction, the first and second lumens being respectively formed at least in part by first and second sides of a first septum.

Clause 2. The catheter according to clause 1, wherein the second lumen includes an inlet port, a first outlet port and a second outlet port, the inlet port of the second lumen being located at a proximal end thereof, the first and second outlet ports being circumferentially spaced-apart from one another with each extending through a wall of the tip at a longitudinal location proximal to the distal end of the tip, located in the second lumen is a second spiral flow or spiral laminar flow inducing structure that is configured to direct a first portion of the fluid towards the first outlet port and a second portion of the fluid to the second outlet port.

Clause 3. The catheter according to clause 2, wherein the second spiral laminar flow inducing structure is configured to cause the first portion of the fluid to spiral in a first rotational direction and to cause the second portion of the fluid to spiral in a second rotational direction opposite the first rotational direction.

Clause 4. The catheter according to clauses 2 and 3, wherein the first and second outlet ports of the second lumen are circumferentially aligned with one another.

Clause 5. The catheter according to any of clauses 2-4, wherein at least a portion of a first structure that forms the first outlet port is configured to induce spiral flow as the first portion of the fluid passes through the first outlet port, and at least a portion of a second structure that forms the second outlet port is configured to induce spiral flow as the second portion of the fluid passes through the second outlet port.

Clause 6. The catheter according to any of clauses 2-5, wherein the second spiral flow or spiral laminar flow inducing structure is a monolithic structure that extends between an inner wall of the second lumen and the first septum that separates at least a portion of a longitudinal length of the first and second lumens.

Clause 7. The catheter according to any of the preceding clauses, wherein the first spiral flow or spiral laminar flow inducing structure is a curved ramp.

Clause 8. The catheter according to any of the preceding clauses, wherein no portion of the first spiral flow or spiral laminar flow inducing structure exists at the inlet port of the first lumen.

Clause 9. The catheter according to any of the preceding clauses, where the second lumen includes a third outlet port, the first, second and third outlet ports of the second lumen being spaced equidistantly apart about a circumference of the tip.

Clause 10. The catheter according to any of the preceding clauses, wherein the tip is a monolithic structure, being made from a single piece of material.

Clause 11. The catheter according to any of the preceding clauses, wherein one or both of the first and second lumens has a non-circular cross section.

Clause 12. The catheter according to any of the preceding clauses, wherein one or both of the first and second lumens is defined at least in part by a straight wall.

### Clause Set B:

Clause 1. A catheter for placement within a vessel or cavity of a body, the catheter comprising:
a tip including:
   a longitudinal axis,
   a first lumen including an inlet port, a first outlet port and a second outlet port, the inlet port being located at a proximal end thereof, the first and second outlet ports being circumferentially spaced-apart from one another with each extending through a wall of the catheter body section at a longitudinal location proximal to the distal end, located in the first lumen is a first spiral flow or spiral laminar flow inducing structure that is configured to direct a first portion of the fluid towards the first outlet port and a second portion of the fluid to the second outlet port,
   a second lumen for carrying the fluid in a proximal direction, at least proximal end portions of the first and second lumens being respectively formed at least in part by first and second sides of a first septum.

Clause 2. The catheter according to clause 1, wherein the second lumen includes an inlet port located at a distal end or distal end portion thereof and an outlet port located at a proximal end or proximal end portion thereof, located inside the second lumen at a first longitudinal distance away from the inlet port is a second spiral laminar flow inducing structure that is located entirely inside and surrounded by the wall or walls that form second lumen.

Clause 3. The catheter according to clause 2, wherein the second spiral flow or spiral laminar flow inducing structure is a curved ramp.

Clause 4. The catheter according to clause 1, wherein at least a distal end portion of the second lumen comprises side-by-side first and second sub-lumens that are separated by a second septum, the first and second sub-lumens respectively including first and second inlet ports located at a distal end or end portion thereof, the second lumen further including at least one outlet port located at a proximal end or end portion thereof, located inside the first sub-lumen at a first longitudinal distance away from the first inlet port is a second spiral flow or spiral laminar flow inducing structure that is located entirely inside and surrounded by the wall or walls that form the first sub-lumen, located inside the second sub-lumen at the first longitudinal distance away from the first inlet port is a third spiral laminar flow inducing structure that is located entirely inside and surrounded by the wall or walls that form the second sub-lumen.

Clause 5. The catheter according to clause 4, wherein each of the second and third spiral flow or spiral laminar flow inducing structures of the second lumen is a curved ramp.

Clause 6. The catheter according to any of clauses 4 and 5, wherein the second spiral flow or spiral laminar flow inducing structure of the first sub-lumen is configured to induce spiral flow or spiral laminar flow in a first rotational direction and the third spiral flow or spiral laminar flow inducing structure of the second sub-lumen is configured to induce spiral flow or spiral laminar flow in a second rotational direct opposite the first rotational direction.

Clause 7. The catheter according to any of clauses 1-6, wherein the first spiral flow or spiral laminar flow inducing structure is configured to cause the first portion of the fluid to spiral in a first rotational direction and to cause the second portion of the fluid to spiral in a second rotational direction opposite the first rotational direction.

Clause 8. The catheter according to any of clauses 1-7, wherein the first and second outlet ports of the first lumen are circumferentially aligned with one another.

Clause 9. The catheter according to any of clauses 1-8, wherein at least a portion of a first structure that forms the first outlet port is configured to induce spiral flow as the first portion of the fluid passes through the first outlet port, and at least a portion of a second structure that forms the second outlet port is configured to induce spiral flow as the second portion of the fluid passes through the second outlet port.

Clause 10. The catheter according to any of clauses 1-9, wherein the first spiral flow or spiral laminar flow inducing structure is a monolithic structure that extends between an inner wall of the first lumen and a septum that separates at least a portion of a longitudinal length of the first and second lumens.

Clause 11. The catheter according to clause 4, wherein the first septum and second septum are arranged perpendicular to one another.

Clause 12. The catheter according to clause 2, wherein no portion of the second spiral flow or spiral laminar flow inducing structure exists at the inlet port of the second lumen.

Clause 13. The catheter according to any of the preceding clauses, where the first lumen includes a third outlet port, the first, second and third outlet ports of the first lumen being spaced equidistantly apart about the circumference of the tip.

Clause 14. The catheter according to any of the preceding clauses, wherein the section of the catheter body is a monolithic structure, being made from a single piece of material.

Clause 15. The catheter according to any of the preceding clauses, wherein one or both of the first and second lumens has a non-circular cross section.

Clause 16. The catheter according to any of the preceding clauses, wherein the one or both of the first and second lumens is defined at least in part by a straight wall.

### Clause Set C:

Clause 1. A catheter for placement within a vessel or cavity of a body, the catheter comprising:

A tip including:
a longitudinal axis,
a first lumen that includes side-by-side first and second sub-lumens that are separated by a first septum, the first and second sub-lumens respectively including first and second inlet ports located at a distal end or distal end portion thereof, the first lumen further including at least one outlet port located at a proximal end or proximal end portion thereof, located inside the first sub-lumen at a first longitudinal distance away from the first inlet port is a first spiral flow or spiral laminar flow inducing structure that is located entirely inside and surrounded by the wall or walls that form first sub-lumen, located inside the second sub-lumen at the first longitudinal distance away from the second inlet port is a second spiral flow or spiral laminar flow inducing structure that is located entirely inside and surrounded by the wall or walls that form second sub-lumen; and
a second lumen for carrying the fluid in a distal direction, at least proximal end portions of the first and second lumens being respectively formed at least in part by first and second sides of a second septum.

Clause 2. The catheter according to clause 1, wherein each of the first and second spiral flow or spiral laminar flow inducing structures of the first lumen is a curved ramp.

Clause 3. The catheter according to any of clauses 1 and 2, wherein the first spiral flow or spiral laminar flow inducing structure of the first sub-lumen is configured to induce spiral flow or spiral laminar flow in a first rotational direction and the second spiral laminar flow inducing structure of the second sub-lumen is configured to induce spiral flow or spiral laminar flow in a second rotational direct opposite the first rotational direction.

Clause 4. The catheter according to clause 1, wherein the first septum and second septum are arranged perpendicular to one another.

Clause 5. The catheter according to any of the preceding clauses, wherein the second lumen includes an inlet port, a first outlet port and a second outlet port, the inlet port of the second lumen being located at a proximal end thereof, the first and second outlet ports being circumferentially spaced-apart from one another with each extending through a wall of the tip at a longitudinal location proximal to the distal end of the tip, located in the second lumen is a third spiral flow or spiral laminar flow inducing structure that is configured to direct a first portion of the fluid towards the first outlet port and a second portion of the fluid to the second outlet port.

Clause 6. The catheter according to clause 5, wherein the third spiral flow or spiral laminar flow inducing structure is configured to cause the first portion of the fluid to spiral in a first rotational direction and to cause the second portion of the fluid to spiral in a second rotational direction opposite the first rotational direction.

Clause 7. The catheter according to clauses 5 and 6, wherein the first and second outlet ports of the second lumen are circumferentially aligned with one another.

Clause 8. The catheter according to any of clauses 5-7, wherein at least a portion of a first structure that forms the first outlet port is configured to induce spiral flow as the first portion of the fluid passes through the first outlet port, and at least a portion of a second structure that forms the second outlet port is configured to induce spiral flow as the second portion of the fluid passes through the second outlet port.

Clause 9. The catheter according to any of clauses 5-8, wherein the third spiral flow or spiral laminar flow inducing structure is a monolithic structure that extends between an inner wall of the second lumen and second septum that separates at least a portion of a longitudinal length of the first and second lumens.

Clause 10. The catheter according to clause 1, wherein no portion of the first spiral flow or spiral laminar flow inducing structure exists at the first inlet port of the first sub-lumen, and no portion of the second spiral flow or spiral laminar flow inducing structure exists at the second inlet port of the second sub-lumen.

Clause 11. The catheter according to clause 5, wherein the second lumen includes a third outlet port, the first, second and third outlet ports of the second lumen being spaced equidistantly apart about the circumference of the tip.

Clause 12. The catheter according to any of the preceding clauses, wherein the tip is a monolithic structure, being made from a single piece of material.

Clause 13. The catheter according to any of the preceding clauses, wherein one or both of the first and second lumens has a non-circular cross section.

Clause 14. The catheter according to any of the preceding clauses, wherein the one or both of the first and second lumens is defined at least in part by a straight wall.

What is claimed is:

1. A catheter for placement within a vessel or cavity of a body of a patient, the catheter comprising:
a tip including:
a longitudinal axis,
a first lumen configured to carry a fluid in a proximal direction through the tip, the first lumen including an inlet port located at a distal end or distal end portion thereof and an outlet port located at a proximal end or a proximal end portion thereof, located inside the first lumen at a first longitudinal distance away from the inlet port is a first spiral flow inducing structure that is located entirely inside and surrounded by a wall or walls that form the first lumen; and
a second lumen configured to carry the fluid in a distal direction through the tip, the first and second lumens being respectively formed at least in part by first and second sides of a first septum.

2. The catheter according to claim 1, wherein the second lumen includes an inlet port, a first outlet port and a second outlet port, the inlet port of the second lumen being located at a proximal end thereof, the first and second outlet ports being circumferentially spaced-apart from one another with each extending through a wall of the tip at a longitudinal location proximal to the distal end of the tip, located in the second lumen is a second spiral flow inducing structure that is configured to direct a first portion of the fluid towards the first outlet port and a second portion of the fluid to the second outlet port.

3. The catheter according to claim 2, wherein the second spiral flow inducing structure is configured to cause the first portion of the fluid to spiral in a first rotational direction and to cause the second portion of the fluid to spiral in a second rotational direction opposite the first rotational direction.

4. The catheter according to claim 2, wherein the first and second outlet ports of the second lumen are circumferentially aligned with one another.

5. The catheter according to claim 2, wherein at least a portion of a first structure that forms the first outlet port is configured to induce spiral flow as the first portion of the fluid passes through the first outlet port, and at least a portion of a second structure that forms the second outlet port is configured to induce spiral flow as the second portion of the fluid passes through the second outlet port.

6. The catheter according to claim 2, wherein the second spiral flow inducing structure is a monolithic structure that extends between an inner wall of the second lumen and the first septum.

7. The catheter according to claim 1, wherein the first spiral flow inducing structure is a curved ramp.

8. The catheter according to claim 1, wherein no portion of the first spiral flow inducing structure exists at the inlet port of the first lumen.

9. The catheter according to claim 2, where the second lumen includes a third outlet port, the first, second and third outlet ports of the second lumen being spaced equidistantly apart about a circumference of the tip.

10. The catheter according to claim 1, wherein the tip is a monolithic structure, being made from a single piece of material.

11. The catheter according to claim 1, wherein the first spiral flow inducing structure is configured to produce a spiral laminar flow of the fluid.

12. The catheter according to claim 2, wherein the second spiral flow inducing structure is configured to direct the first portion of the fluid towards the first outlet port with laminar flow and a second portion of the fluid to the second outlet port with laminar flow.

13. A catheter for placement within a vessel or cavity of a body of a patient, the catheter comprising:
a tip including:
a longitudinal axis,
a first lumen including an inlet port, a first outlet port and a second outlet port, the inlet port being located at a proximal end thereof, the first and second outlet ports being circumferentially spaced-apart from one another with each extending through a wall of the tip at a longitudinal location proximal to the distal end of the catheter body section, located in the first lumen is a first spiral flow inducing structure that is configured to direct a first portion of the fluid towards the first outlet port and a second portion of the fluid to the second outlet port; and
a second lumen for carrying the fluid in a proximal direction, at least proximal end portions of the first and second lumens being respectively formed at least in part by first and second sides of a first septum.

14. The catheter according to claim 13, wherein at least a distal end portion of the second lumen comprises side-by-side first and second sub-lumens that are separated by a second septum, the first and second sub-lumens respectively including first and second inlet ports located at a distal end or end portion thereof, the second lumen further including at least one outlet port located at a proximal end or end portion thereof, located inside the first sub-lumen at a first longitudinal distance away from the first inlet port is a second spiral flow inducing structure that is located entirely inside and surrounded by a wall or walls that form the first sub-lumen, located inside the second sub-lumen at the first longitudinal distance away from the first inlet port is a third spiral laminar flow inducing structure that is located entirely inside and surrounded by a wall or walls that form the second sub-lumen.

15. The catheter according to claim 14, wherein each of the second and third spiral flow inducing structures is a curved ramp.

16. The catheter according to claim 14, wherein the second spiral flow inducing structure of the first sub-lumen is configured to induce spiral flow in a first rotational direction and the third spiral flow inducing structure of the second sub-lumen is configured to induce spiral flow in a second rotational direct opposite the first rotational direction.

17. The catheter according to claim 1, wherein the first spiral flow inducing structure is configured to cause the first portion of the fluid to spiral in a first rotational direction and to cause the second portion of the fluid to spiral in a second rotational direction opposite the first rotational direction.

18. The catheter according to claim 1, wherein at least a portion of a first structure that forms the first outlet port is configured to induce spiral flow as the first portion of the fluid passes through the first outlet port, and at least a portion of a second structure that forms the second outlet port is configured to induce spiral flow as the second portion of the fluid passes through the second outlet port.

19. The catheter according to claim 1, wherein the catheter is a dialysis catheter and the fluid is blood.

20. The catheter according to claim 13, wherein the catheter is a dialysis catheter and the fluid is blood.
